(19) United States
(12) Patent Application Publication
(10) Pub. No.: US 2010/0076070 A1
(43) Pub. Date: Mar. 25, 2010

(54) Forskolin Compositions and Methods For Administration

(75) Inventor: William J. Roberts, Ocala, FL (US)

Correspondence Address: Don D. Cha, 547 Buena Vista Road, Golden, CO, 80401, US

(73) Assignee: BIOTEST LABORATORIES, LLC., Colorado Springs, CO (US)

(21) Appl. No.: 12/626,717

(22) Filed: Nov. 27, 2009

Related U.S. Application Data
(63) Continuation of application No. 10/883,995, filed on Jul. 1, 2004, now abandoned.

(51) Int. Cl. A61K 31/352; A61P 3/00

(52) U.S. Cl. 514/455

(57) ABSTRACT

An administration method is provided, in which a composition comprising a forskolin 1α,9α-carbonate compound is administered to a human subject. According to an embodiment, the forskolin 1α,9α-carbonate compound has a skeletal structure of 8,13-epoxy-1α,6β,7β,9α-tetrahydroxylabd-14-en-11-one. A carbonyl group links the 1-position hydroxy oxygen and the 9-position hydroxy oxygen to one another for forming a carbonate ester ring. Hydrogen is appended to the 6-position hydroxy oxygen. A hydrogen or an acetyl group is appended to the 7-position hydroxy oxygen.

### BACKGROUND OF THE INVENTION

1. Field of the Invention

The present invention pertains to the field of biochemistry and pharmacology and, more specifically, to compositions and related methods for addressing fat loss and/or depression in human subjects, and potentially in animals.

2. Description of Related Art

Forskolin, sometimes referred to as forskohlin, and also known as 8,13-epoxy-1α,6β,7β,9α-tetrahydroxylabd-14-en-11-one 7β-acetate, is a diterpenoid compound having the following chemical structure:

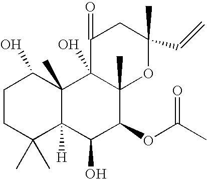

The numbering system for the carbon skeleton is well known to those in the art, and is disclosed, for example, in U.S. Pat. No. 4,088,659.

Forskolin is generally obtained by extraction from the plant known as coleus forskohlii. Traditional Ayurvedic medicine has used herbal preparations from coleus forskohlii for various therapeutic and medicinal purposes, including the treatment of heart and lung diseases, intestinal spasms, insomnia, convulsions, uterine cramps, painful urination, angina, and hypertension.

Forskolin is also reported to possess therapeutic and medicinal properties for treating many of the infirmities conventionally treated by coleus forskohlii. In addition, forskolin also reportedly has been used for other therapeutic purposes, such as ophthalmic (for lowering eye pressure and reducing the risk of glaucoma), asthma and other allergic conditions, and psoriasis, to name a few. Forskolin also has been proposed for clinical applications including weight-loss programs, hypothyroidism, malabsorption and digestive disorders, depression, prevention of cancer metastases, and immune system enhancement.

Without wishing to be bound by any theory, it is believed that forskolin activates the enzyme adenylate cyclase. Adenylate cyclase regulates the formation of cyclic adenosine monophosphate (cAMP), a compound that in turn exerts control over numerous cell activities. Once adenylate cyclase has been activated, the intracellular levels of cAMP are raised. Cyclic AMP activates many other enzymes involved in diverse cellular functions. Under normal circumstances, cAMP is formed when a stimulatory hormone, such as epinephrine, binds to a receptor site on the cell membrane and stimulates the activation of adenylate cyclase. Forskolin is believed to by-pass this need for direct hormonal activation of adenylate cyclase via transmembrane activation.

The physiological and biochemical effects of a raised intracellular cAMP level include inhibition of platelet activation and degranulation; inhibition of mast cell degranulation and histamine release; increased force of contraction of heart muscle; relaxation of arteries and other smooth muscles; increased insulin secretion; increased thyroid function; and increased lipolysis.

Recent studies have concluded that forskolin possesses additional mechanisms of action independent of its ability to directly stimulate adenylate cyclase and cAMP-dependent physiological responses. Specifically, forskolin has been reported to inhibit a number of membrane transport proteins and channel proteins through a mechanism that does not involve the production of cAMP. Another action of forskolin is on antagonizing the action of platelet-activating factor (PAF) by interfering with PAF binding to receptor sites. PAF plays a central role in many inflammatory and allergic processes, including neutrophil activation, increased vascular permeability, smooth muscle contraction including bronchoconstriction, and reduction in coronary blood flow.

Problematically, forskolin is believed to have relatively low bioavailability in human subjects when taken orally. Further, forskolin is believed to have an undesirably short duration of action, which may be only a few hours or less. In attempts to address these problems, numerous prodrugs of forskolin have been proposed in, for example, Sujata Bhat et al., The Antihypertensive and Positive Inotropic Diterpene Forskolin: Effects of Structural Modifications on its Activities, J. Med. Chem. 26, 486-492 (1983) (hereinafter “Bhat”), and J. Chem. So., Perkin Trans 1, 767 (1982). It is generally known in the art that in some instances, but not in others, prodrug derivitization of compounds may improve oral bioavailability and duration of action in human subjects. Prodrug derivitization is known in the art and referred to herein as the addition of one or more labile chemical groups (or promoieties) as substitute(s) for one or more atoms of the parent molecule. Bhat, however, has reported that forskolin prodrugs do not provide advantages over forskolin.

### OBJECTS OF THE INVENTION

Accordingly, an object of the present invention is to provide compositions and methods that can be used to promote fat loss in human subjects.

Another object of the invention is to provide compositions and methods that can be used to inhibit depression in human subjects.

Another object of the invention is to provide compositions and methods that can increase the in vivo concentration, bioavailability, and/or duration of action of forskolin-related compounds.

Another object of the invention according to certain aspects is to provide compounds and methods that can be used to increase the in vivo concentration and bioavailability of forskolin-related compounds while being amenable to convenient administration, such as oral administration.

Additional objects and advantages of the invention will be set forth in the description that follows, and in part will be apparent from the description, or may be learned by practice of the invention. The objects and advantages of the invention may be realized and obtained by means of the instrumentalities and combinations pointed out in the appended claims.

### SUMMARY OF THE INVENTION

To achieve the foregoing objects, and in accordance with the purposes of the invention as embodied and broadly described in this document, a method is provided for administration of a composition to a human subject to promote fat loss. The method comprises providing a composition comprising a forskolin 1α,9α-carbonate compound. The forskolin 1α,9α-carbonate compound comprises a skeletal structure of 8,13-epoxy-1α,6β,7β,9α-tetrahydroxylabd-14-en-11-one, wherein the skeletal structure has a 1-position hydroxy oxygen appended to a 1-position carbon, a 6-position hydroxy oxygen appended to a 6-position carbon, a 7-position hydroxy oxygen appended to a 7-position carbon, and a 9-position hydroxy oxygen appended to a 9-position carbon. The forskolin 1α,9α-carbonate compound also comprises a carbonyl group linking the 1-position hydroxy oxygen and the 9-position hydroxy oxygen to one another to form a carbonate ester ring. In addition, it comprises a hydrogen appended to the 6-position hydroxy oxygen, and an acetyl group (—C(O)CH₃) appended to the 7-position hydroxy oxygen. The method further comprises administering the composition to the human subject.

The providing of the composition preferably but optionally comprises providing the composition to include a lauroyl macrogol-32 glyceride. The administration of the composition to the human subject preferably comprises administering the composition so that the forskolin 1α,9α-carbonate compound is in a dosage range of about 10 mg/day to about 80 mg/day, more preferably about 20 mg/day to about 60 mg/day, and more preferably about 30 mg/day. The administration of the composition to the human subject in preferred implementations comprises administering the composition in two separate dosages daily. In the circumstance in which two such dosages are administered daily, each of the two separate dosages preferably comprises about 20 mg to about 40 mg of the forskolin 1α,9α-carbonate compound.

The forskolin 1α,9α-carbonate compound may be made into a micronized form. It may be administered through oral ingestion of the composition, although other routes of administration are possible. In accordance with another aspect of the invention, a method is provided for administration of a composition to a human subject to inhibit depression. The method comprises providing a composition comprising a forskolin 1α,9α-carbonate compound. The forskolin 1α,9α-carbonate compound comprises a skeletal structure of 8,13-epoxy-1α,6β,7β,9α-tetrahydroxylabd-14-en-11-one, wherein the skeletal structure has a 1-position hydroxy oxygen appended to a 1-position carbon, a 6-position hydroxy oxygen appended to a 6-position carbon, a 7-position hydroxy oxygen appended to a 7-position carbon, and a 9-position hydroxy oxygen appended to a 9-position carbon. The forskolin 1α,9α-carbonate compound also comprises a carbonyl group linking the 1-position hydroxy oxygen and the 9-position hydroxy oxygen to one another to form a carbonate ester ring. In addition, it comprises a hydrogen appended to the 6-position hydroxy oxygen, and an acetyl group (—C(O)CH₃) appended to the 7-position hydroxy oxygen. The method further comprises administering the composition to the human subject. The detailed description of the composition and of applicable and preferred dosages as described above apply in this method as well.

In accordance with another aspect of the invention, a composition is provided which comprises a forskolin 1α,9α-carbonate compound. The forskolin 1α,9α-carbonate compound comprises a skeletal structure of 8,13-epoxy-1α,6β,7β,9α-tetrahydroxylabd-14-en-11-one, wherein the skeletal structure having a 1-position hydroxy oxygen appended to a 1-position carbon, a 6-position hydroxy oxygen appended to a 6-position carbon, a 7-position hydroxy oxygen appended to a 7-position carbon, and a 9-position hydroxy oxygen appended to a 9-position carbon. The forskolin 1α,9α-carbonate compound comprises a carbonyl group linking the 1-position hydroxy oxygen and the 9-position hydroxy oxygen to one another to form a carbonate ester ring, a hydrogen appended to the 6-position hydroxy oxygen, and an acetyl group (—C(O)CH₃) appended to the 7-position hydroxy oxygen. The composition further comprises a lauroyl macrogol-32 glyceride.

In accordance with another aspect of the invention, a method is provided for administration of a composition to a human subject to promote fat loss. The method comprises providing a composition comprising a forskolin 1α,9α-carbonate compound, wherein this compound comprises a skeletal structure of 8,13-epoxy-1α,6β,7β,9α-tetrahydroxylabd-14-en-11-one, and wherein the skeletal structure has a 1-position hydroxy oxygen appended to a 1-position carbon, a 6-position hydroxy oxygen appended to a 6-position carbon, a 7-position hydroxy oxygen appended to a 7-position carbon, and a 9-position hydroxy oxygen appended to a 9-position carbon. The forskolin 1α,9α-carbonate compound comprises a carbonyl group linking the 1-position hydroxy oxygen and the 9-position hydroxy oxygen to one another to form a carbonate ester ring, a hydrogen appended to the 6-position hydroxy oxygen, and a hydrogen appended to the 7-position hydroxy oxygen. The method also includes administering the composition to the human subject. Dosages and optional aspects of the above-mentioned methods and compounds would apply here as well.

In accordance with yet another aspect of the invention, a method is provided for administration of a composition to a human subject to inhibit depression. The method comprises providing a composition comprising a forskolin 1α,9α-carbonate compound comprising a skeletal structure of 8,13-epoxy-1α,6β,7β,9α-tetrahydroxylabd-14-en-11-one. The skeletal structure has a 1-position hydroxy oxygen appended to a 1-position carbon, a 6-position hydroxy oxygen appended to a 6-position carbon, a 7-position hydroxy oxygen appended to a 7-position carbon, and a 9-position hydroxy oxygen appended to a 9-position carbon. The forskolin 1α,9α-carbonate compound comprises a carbonyl group linking the 1-position hydroxy oxygen and the 9-position hydroxy oxygen to one another to form a carbonate ester ring, a hydrogen appended to the 6-position hydroxy oxygen, and a hydrogen appended to the 7-position hydroxy oxygen. The method also includes administering the composition to the human subject. The dosages and modifications apply here as well.

In accordance with still another aspect of the invention, a composition is provided that comprises a forskolin 1α,9α-carbonate compound. The forskolin 1α,9α-carbonate compound comprises a skeletal structure of 8,13-epoxy-1α,6β,7β,9α-tetrahydroxylabd-14-en-11-one. The skeletal structure has a 1-position hydroxy oxygen appended to a 1-position carbon, a 6-position hydroxy oxygen appended to a 6-position carbon, a 7-position hydroxy oxygen appended to a 7-position carbon, and a 9-position hydroxy oxygen appended to a 9-position carbon. The forskolin 1α,9α-carbonate compound comprises a carbonyl group linking the 1-position hydroxy oxygen and the 9-position hydroxy oxygen to one another to form a carbonate ester ring, a hydrogen appended to the 6-position hydroxy oxygen, and a hydrogen appended to the 7-position hydroxy oxygen. The composition further includes a lauroyl macrogol-32 glyceride.

In accordance with another aspect of the invention, a method is provided for administration of a composition to a human subject to promote fat loss. The method comprises providing a composition comprising a forskolin 1α,9α-carbonate compound comprising a skeletal structure of 8,13-epoxy-1α,6β,7β,9α-tetrahydroxylabd-14-en-11-one, the skeletal structure having a 1-position hydroxy oxygen appended to a 1-position carbon, a 6-position hydroxy oxygen appended to a 6-position carbon, a 7-position hydroxy oxygen appended to a 7-position carbon, and a 9-position hydroxy oxygen appended to a 9-position carbon, a first carbonyl group linking the 1-position hydroxy oxygen and the 9-position hydroxy oxygen to one another to form a first carbonate ester ring, and a second carbonyl group linking the 6-position hydroxy oxygen and the 7-position hydroxy oxygen to one another to form a second carbonate ester ring. The method also includes administering the composition to the human subject. The modifications and dosages noted above apply to this method as well.

In accordance with yet another aspect of the invention, a method is provided for administration of a composition to a human subject to inhibit depression. The method comprises providing a composition comprising a forskolin 1α,9α-carbonate compound comprising a skeletal structure of 8,13-epoxy-1α,6β,7β,9α-tetrahydroxylabd-14-en-11-one. The skeletal structure has a 1-position hydroxy oxygen appended to a 1-position carbon, a 6-position hydroxy oxygen appended to a 6-position carbon, a 7-position hydroxy oxygen appended to a 7-position carbon, and a 9-position hydroxy oxygen appended to a 9-position carbon. The forskolin carbonate compound comprises a first carbonyl group linking the 1-position hydroxy oxygen and the 9-position hydroxy oxygen to one another to form a first carbonate ester ring, and a second carbonyl group linking the 1-position hydroxy oxygen and the 9-position hydroxy oxygen to one another to form a second carbonate ester ring. The method further includes administering the composition to the human subject. The modifications and dosages noted above apply here also.

In accordance with another aspect of the invention, a composition is provided that comprises a forskolin 1α,9α-carbonate compound comprising a skeletal structure of 8,13-epoxy-1α,6β,7β,9α-tetrahydroxylabd-14-en-11-one, wherein the skeletal structure has a 1-position hydroxy oxygen appended to a 1-position carbon, a 6-position hydroxy oxygen appended to a 6-position carbon, a 7-position hydroxy oxygen appended to a 7-position carbon, and a 9-position hydroxy oxygen appended to a 9-position carbon. The forskolin 1α,9α-carbonate compound comprises a first carbonyl group linking the 1-position hydroxy oxygen and the 9-position hydroxy oxygen to one another to form a first carbonate ester ring, and a second carbonyl group linking the 1-position hydroxy oxygen and the 9-position hydroxy oxygen to one another to form a second carbonate ester ring. The composition further includes a lauroyl macrogol-32 glyceride.

### DETAILED DESCRIPTION OF THE PREFERRED EMBODIMENTS AND METHODS

Reference will now be made in detail to the presently preferred embodiments and methods of the invention. It should be noted, however, that the invention in its broader aspects is not limited to the specific details, representative compositions and methods, and illustrative examples described in this section in connection with the preferred embodiments and methods. The invention according to its various aspects is particularly pointed out and distinctly claimed in the attached claims read in view of this specification, and appropriate equivalents.

It is to be noted that, as used in the specification and the appended claims, the singular forms “a,” “an,” and “the” include plural referents unless the context clearly dictates otherwise.

In accordance with one aspect of the invention, a method is provided for the administration of a composition to a human subject. The compound preferably but optionally is for administration to a human being. This is not necessarily limiting, however, and veterinary applications also are possible in certain instances. The administration preferably is to promote fat loss in the subject, and/or to inhibit depression in the subject, although other effects are possible.

The method comprises providing a composition that comprises a forskolin 1α,9α-carbonate. This numbering corresponds to the ring identification and carbon numbering system well known in the field of organic chemistry. It is illustrated in U.S. Pat. No. 4,088,659, as noted above. The numbering of positions on the compound as they are provided in this document utilize that number scheme. The forskolin 1α,9α-carbonate compound comprises a skeletal structure of 8,13-epoxy-1α,6β,7β,9α-tetrahydroxylabd-14-en-11-one. The forskolin 1α,9α-carbonate compound comprises a skeletal structure including carbon atoms at the 1 position, 6 position, 7 position, and 9 position, respectively. Appended to the 1-position carbon, 6-position carbon, 7-position carbon, and the 9-position carbon are a 1-position oxygen, 6-position oxygen, 7-position oxygen, and a 9-position oxygen, respectively. These oxygen atoms are also referred to herein as a 1-position hydroxy oxygen, a 6-position hydroxy oxygen, a 7-position hydroxy oxygen, and a 9-position hydroxy oxygen, respectively. The term “hydroxy oxygen” is to some extent a misnomer as used in this application, inasmuch as the forskolin 1α,9α-carbonate compound according to this invention does not necessarily include hydrogen atoms respectively appended directly to the 1-position, 6-position, 7-position, and 9-position hydroxy oxygen atoms. For example, forskolin 1α,9α-carbonate has carbonyl groups appended to the 1 and 9 position oxygen atoms.

The forskolin 1α,9α-carbonate compound according to this aspect of the invention further comprises a carbonyl group (—C(O)—) linking the 1-position hydroxy oxygen and the 9-position hydroxy oxygen to one another to form a carbonate ester ring. Preferably, the carbonate ester ring comprises, clockwise, the carbonyl carbon, the 9-position hydroxy oxygen, the 9-position carbon, a 10-position carbon, the 1-position carbon, and the 1-position hydroxy oxygen.

A hydrogen atom is appended to the 6-position hydroxy oxygen, i.e., forming a hydroxyl group. The 7-position hydroxy oxygen can be appended to two different constituents, and these constituents thus may be represented by an —R, as shown in Formula I below.

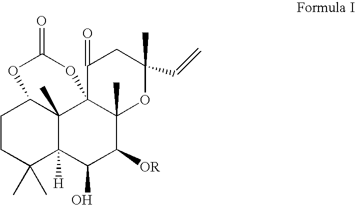

In one presently preferred embodiment, R is an acetyl group (—C(O)CH₃), as shown in Formula II below.

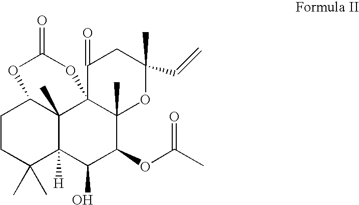

In another presently preferred embodiment, R is a hydrogen atom or proton. This compound is also referred to herein as 7-deacetyl forskolin 1α,9α-carbonate, or simply “7-DAF.”

In accordance with another aspect of the invention, another method is provided for the administration of a composition to a human subject to promote fat loss, and/or for treatment and inhibition of depression. The method comprises providing a composition comprising a forskolin 1α,9α-carbonate compound comprising a skeletal structure of 8,13-epoxy-1α,6β,7β,9α-tetrahydroxylabd-14-en-11-one, wherein the skeletal structure has a 1-position hydroxy oxygen appended to a 1-position carbon, a 6-position hydroxy oxygen appended to a 6-position carbon, a 7-position hydroxy oxygen appended to a 7-position carbon, and a 9-position hydroxy oxygen appended to a 9-position carbon. The forskolin 1α,9α-carbonate compound comprises a first carbonyl group linking the 1-position hydroxy oxygen and the 9-position hydroxy oxygen to one another to form a first carbonate ester ring, and a second carbonyl group linking the 6-position hydroxy oxygen and the 7-position hydroxy oxygen to one another to form a second carbonate ester ring. The method further includes administering the composition to the human subject.

The compositions as described herein above, including the forskolin 1α,9α-carbonate compounds, comprise yet another aspect of the invention.

In accordance with the methods pursuant to these aspects of the invention, the composition comprising the forskolin carbonate is administered to the subject. This administration preferably comprises oral administration, although transdermal administration and/or other means may be suitable. The administration of the composition also may be by combinations of these techniques or approaches.

As part of the method, the administration of the composition may comprise complexing the composition with pharmaceutically acceptable excipients, such as powdered cellulose, and/or agents to facilitate bioabsorption and the like. The composition administration optionally may further combine the forskolin carbonate compound with a suitable carrier prior to administering the composition to a subject. A presently preferred example would include a lauroyl macrogol-32 glyceride, for example, such as Gelucire 44/14, commercially available from Gattefosse Corporation.

One drawback of many known compositions of this general type is that absorption may be inferior or not superior to the parent compound, or conversion to the parent compound may be incomplete, resulting in reduced efficacy compared to the parent compound. Another drawback to known compositions of this type, especially esters, in oral administration is that the promoeity may be removed by hydrolysis in the gastrointestinal tract. Yet another drawback that exists with many compositions of this type is an inappropriate or undesirable duration of action, generally speaking, a duration of less than about 8 hours or greater than 24 hours. Durations of less than 8 hours are disadvantageous because administration more than three times a day is inconvenient for the user, and can lead to attenuated levels if one or more daily dosages are missed. Durations substantially in excess of 24 hours are disadvantageous because it may take several days of administration for a level state to be achieved, and because such durations create problems in establishing a consistent dosage routine. For these reasons, most compounds administered as drugs are not administered as prodrugs.

The compositions comprising forskolin 1α9α-carbonate compounds according to embodiments of the invention can be particularly useful, when taken orally, in aiding fat loss, as well as in the treatment of depression and inhibition of it. Inhibition of depression as referred to herein means any tendency to lessen the effects of depression, and does not necessarily refer to complete eradication of the depression. Other oral applications include, for example and not necessarily by limitation, for treating angina, treating hypertension, increasing force of heart contractions, treating congestive heart failure, decreasing platelet aggregation, achieving relaxation of arteries, achieving relaxation of smooth muscles, enhancing the immune system, increasing insulin secretion, increasing thyroid function, treating menstrual cramps, and treating painful urination. Topically, the forskolin 1α9α-carbonate compound of embodiments of the invention may be used, for example, in the treatment of psoriasis.

The preferred dosage of the compound will depend upon the subject or class of subject to which the composition is to be administered, the desired effects to be achieved, and other factors commonly affecting dosage determinations for this type of composition. In accordance with presently preferred versions of the inventive compositions and methods, as described above for the various aspects of the invention, the amount or dosage of the composition that is administered to the subject is effective to achieve the desired result, i.e., to promote fat loss where that is the objective, and to inhibit depression where that is the objective.

In accordance with these aspects of the invention, the preferred methods of administration may have daily dosages of the composition so that the forskolin 1α,9α-carbonate compound is in a dosage range of about 10 mg/day to about 80 mg/day. More preferably, the dosage range for the forskolin 1α,9α-carbonate compound is about 20 mg/day to about 60 mg/day. Still more preferably, the daily dosage for the forskolin 1α,9α-carbonate compound is in about 30 mg/day.

The compound administration may involve administering the compound once per day, or may be divided into a plurality of dosages given throughout the day. In the presently preferred implementation of the method, the administration of the composition to the human subject comprises administering the composition in two separate dosages daily, i.e., two or more dosages during a 24-hour period. Preferably in instances where two dosages are given daily, the administration of the composition to the human subject comprises administering the composition so that each of the two separate dosages comprises about 20 mg to about 40 mg of the forskolin 1α,9α-carbonate compound. One or two administrations per day is/are preferred. In the event that compound is administered twice daily, the dosages are preferably 8-16 hours apart, more preferably 10-14 hours apart, and more preferably are given at 12-hour intervals.

The forskolin 1α9α-carbonate compound of embodiments of the invention may be produced in the following forms: an ordinary or micronized powder; powder combined with cellulose and/or other pharmaceutically acceptable powdered excipient; or in oil solution. As mentioned above, the forskolin carbonate compound optionally may be complexed, for example, with pharmaceutically acceptable excipients, such as powdered cellulose. The compound may also be administered with a carrier, which may comprise a solid carrier, a semi-solid carrier, or a liquid carrier. The capsules may be filled to a volume of from 0.33 to 0.80 mL preferably.

Forskolin carbonate may also be provided in a self-emulsifying microemulsion formulation, for example, comprising a mixture of a cosurfactant with hydrophilic/lipophilic balance (HLB) of approximately 5 or 6, and a surfactant with a HLB of approximately 14 or 15, in a weight/weight ratio of, for example, approximately 57/43. The solubility of forskolin carbonate in this formulation is preferably at least 20 mg/mL, and dosing may be up to 20 mg per capsule or more, more preferably 5 to 20 mg forskolin carbonate per capsule. Preferred co-surfactants are propylene glycol fatty acid esters with a hydrophilic/lipophilic balance of approximately 5 or 6, and more specifically propylene glycol caprylate esters. This also includes propylene glycol esters that are principally monoesters, for example, 60% or 90%. Examples would include Capryol PGMC and/or Capryol 90 each manufactured by Gattefosse Corporation. Another preferred co-surfactant is Plurol Oleique, also manufactured by Gattefosse Corporation. Plurol Oleique may be used in a microemulsion formulation as a less preferred excipient (combination of co-surfactant and surfactant), or may be used by itself or with an oil as an oily carrier without any other surfactant as excipient. A preferred surfactant is polyoxyl caster oil, such as Cremophor EL and/or Cremophor RH, manufactured by BASF. The excipient is combined with forskolin carbonate and any other drug or nutritional supplement products that may be desired to be simultaneously delivered, using about 0.700 to 0.850 mL per capsule. A suitable capsule type is Licaps by Capsugel Corporation. Preferred sizes for doses of 10 mg per capsule are single-ought (0) or double-ought (00). For doses of 5 mg per capsule, size 1 or size 2 are preferred, although sizes 0 and 00 are also acceptable. Additionally but less preferably, softgel capsules may be used. Self-emulsifying microemulsion formulations and methods for making the same are disclosed in U.S. Pat. Nos. 6,054,136 and 6,312,704, the disclosures of which are incorporated herein by reference.

An example of another embodiment comprises forskolin carbonate in a self-emulsifying microemulsion formulation comprising about 50 weight percent of at least one member selected from Capryol PGMC, Capryol 90, and Plurol Oleique, and about 50 weight percent of at least one member selected from a polyethylene glycol-15-hydroxystearate, such as Solutol HS-15, manufactured by BASF. According to yet another embodiment, the forskolin carbonate is in a self-emulsifying microemulsion formulation comprising a lauroyl macrogol-32 glyceride, such as Gelucire 44/14, manufactured by Gattefosse Corp. Where Gelucire 44/14 is selected, heating is preferred to melt the excipient and mix with active ingredients, e.g., the forskolin carbonate, and to fill the capsule. An appropriate heating temperature is 50° C. to 55° C., with a maximum of about 70° C. Similarly, when Plurol Oleique and/or Cremophor RH is/are used, heating is preferred to mix the formulation and fill the capsules. Appropriate heating temperatures include 35° C. to 55° C., with a maximum of about 70° C. Temperatures may also be limited or influenced by the capsule (e.g., Licaps) selected.

Forskolin carbonate also may be provided in different self-emulsifying microemulsion formulations, for example, comprising a mixture of a surfactant with a hydrophilic/lipophilic balance (HLB) of approximately 10 or greater, preferably 14 or 15, combined with a cosurfactant with a HLB of approximately 5 to 9, optionally with a lipoidal phase of HLB of 4 or less.

According to another aspect of the invention, a method is provided for making the above-mentioned forskolin 1α,9α-carbonate compounds.

Forskolin (8,13-epoxy-1α,6β,7β,9α-tetrahydroxylabd-14-en-11-one 7β-acetate) may be obtained commercially, e.g., from Sigma Aldrich and Sabinsa. Alternatively, forskolin may be derived from coleus forskohlii according to known methods, such as described in U.S. Pat. No. 4,088,659, or may be synthesized by known methods, including, for example, those described in S. Hashimoto et al., Journal of American Chemical Society, 110, 3670.

According to a preferred yet optional embodiment of this aspect of the invention, the 1α,9α-carbonate group may be attached to the forskolin skeletal structure by reacting forskolin (7β-acetyl-8,13-epoxy-1α,6β,7β,9α-tetrahydroxylabd-14-en-11-one) or deacetyl forskolin (7β-deacetyl-8,13-epoxy-1α,6β,7β,9α-tetrahydroxylabd-14-en-11-one) with phosgene (Cl—C(O)—Cl) to produce 7β-acetyl-8,13-epoxy-1α,6β,7β,9α-tetrahydroxylabd-14-en-11-one 1α,9α-carbonate or 7β-deacetyl-8,13-epoxy-1α,6β,7β,9α-tetrahydroxylabd-14-en-11-one 1α,9α-carbonate, respectively. Phosgene preferably is dissolved in a suitable carrier, such as, for example, toluene or tetrahydrofuran (THF). As an alternative to phosgene, trichloromethyl chloroformate or the like may also be used. The reaction may optionally be carried out in the presence of a catalyst, such as activated carbon. Further details concerning suitable reaction conditions may be found in Wu, Pei-Lin, et al, Journal of the Chinese Chemical Society, Vol. 47, pp. 271-274 (2000), which discusses method for making chloroformates. Because of the high reactivity of phosgene, the reaction is preferably carried out at a relatively low temperature, such as, for example, about 0° C. Reaction at the 1,9-hydroxy oxygen atoms is more selective at cold temperatures, reducing by-products and unwanted reactions. Extraction of the resulting forskolin 1α,9α-carbonate compound may be carried out in a suitable solvent system, for example, such as hexane, cyclohexane, pentane, ethyl acetate, ether, petroleum ether, chloroform, or combinations of these. The product may be diluted with water after the reaction with phosgene has reached completion for neutralization purposes, and dried, for example, with sodium sulfate or otherwise (e.g., vacuum).

In the event a different substituent other than an acetyl group is desired at the 7-position oxygen, the 7-position acetyl may be cleaved from the 7-position hydroxy oxygen, for example, via acid or alkaline hydrolysis, optionally in the presence of an organic cosolvent. The cleaving agent preferably does not react with the 11-keto group. For example, diisopropyl amine may be selected as the cleaving agent. The appropriate substituent optionally may then be appended onto the 7-position hydroxy oxygen.

The following non-exhaustive, illustrative example further explains the methods and principles of the present invention.

### Example

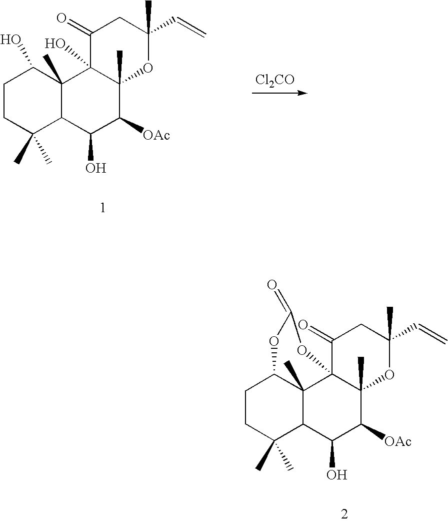

To a flask fitted with a magnetic stirrer, forskolin (25 g, 63 mmol) and anhydrous pyridine were added under argon atmosphere. The mixture was stirred at 0° C., and phosgene (20% solution in toluene, 50 mL, 100 mmol) was added dropwise for 30 min. The mixture was stirred at room temperature for 4 hours, cooled to 0° C., and water (100 mL) was added dropwise. The resulting mixture was diluted with water (100 mL) and extracted with ethyl acetate (100 mL). The organic phase was separated, washed with 5% aq HCl (2×100 mL), and additional ethyl acetate (300 mL) was added. The solution was washed with 5% NaCl (400 mL), saturated NaCl (200 mL) and dried over anhydrous Na₂SO₄. The solution was concentrated under reduced pressure to give bulk crystallization of the product, and hexane (200 mL) was slowly added. The mixture was stirred for 3 h, the product was collected by filtration, and dried in vacuum. This yielded 22.7 g (86%) of forskolin carbonate material.

Additional advantages and modifications will readily occur to those skilled in the art. Therefore, the invention in its broader aspects is not limited to the specific details, representative devices and methods, and illustrative examples shown and described. Accordingly, departures may be made from such details without departing from the spirit or scope of the general inventive concept as defined by the appended claims and their equivalents.

1-57. (canceled)

58. A method for reducing the amount of fat in a human subject, said method comprising administering to the subject an effective amount of a composition comprising a forskolin 1α,9α-carbonate compound to reduce the amount of fat in the subject, wherein the forskolin 1α,9α-carbonate compound is of the formula: 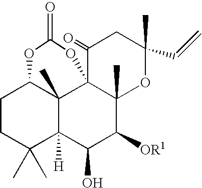
wherein R¹ is hydrogen or a carbonyl group.

59. The method of claim 59, wherein R¹ is a carbonyl group.

60. The method of claim 59, wherein R¹ is hydrogen or an acetyl group.

61. The method of claim 60, wherein R¹ is hydrogen.

62. The method of claim 60, wherein R¹ is an acetyl group.

63. The method of claim 58, wherein the composition further comprises a lauroyl macrogol-32 glyceride.

64. The method of claim 58, wherein the amount of forskolin 1α,9α-carbonate compound administered range from about 10 mg/day to about 80 mg/day.

65. The method of claim 58, wherein the amount of forskolin 1α,9α-carbonate compound administered range from about 20 mg/day to about 60 mg/day.

66. The method of claim 58, wherein the composition is administered orally.
